(19) United States
(12) Patent Application Publication
(10) Pub. No.: US 2008/0102057 A1
(43) Pub. Date: May 1, 2008

(54) BIPOTENTIAL LIVER CELL LINES FROM WILD-TYPE MAMMALIAN LIVER TISSUE

(75) Inventors: Helene Strick-Marchand, Paris (FR); Mary Weiss, Paris (FR); Serban Morosan, Paris (FR); Dina Kremsdorf, Paris (FR); Pierre Charneau, Paris (FR)

Correspondence Address: OBLON, SPIVAK, MCCLELLAND MAIER & NEUSTADT, P.C., 1940 DUKE STREET, ALEXANDRIA, VA, 22314, US

(73) Assignees: INSERM, Paris (FR); Centre National De La Recherche Scient., Paris Cedex 16 (FR); INSTITUT PASTEUR, Paris (FR)

(21) Appl. No.: 11/969,481

(22) Filed: Jan. 4, 2008

Related U.S. Application Data
(63) Continuation of application No. 10/677,303, filed on Oct. 3, 2003, now abandoned.

(51) Int. Cl. A61K 48/00; A01N 1/02; A61P 43/00; C12N 5/00; C12N 5/02; G01N 33/53

(52) U.S. Cl. 424/93.2; 435/1.1; 435/325; 435/354; 800/8

(57) ABSTRACT

The present invention relates to a hepatic cell line derived from wild-type mammalian liver tissue by culture methodology, the cells of the cell line being capable of differentiating into hepatocytes and bile duct cells. The present invention also relates to methods of producing such cells as well as to their applications in therapy and as an investigational tool.

### BACKGROUND OF THE INVENTION

1. Field of the Invention

The present invention relates to cultured liver cells, culturing methods as well as to their applications in therapy and as an investigational tool.

2. Description of the Background

Bipotential hepatoblasts are first observed in the embryo, following liver bud formation at around day 10 of gestation (E10) in the mouse. Hepatoblasts begin to differentiate at E14 into the two major cell types of the liver: hepatocytes and bile duct cells (cholangiocytes). Hepatoblasts express liver-enriched transcription factors (LETF), α-fetoprotein (AFP), albumin, cytokeratins (CK) 8 and 18 but not markers of mature hepatocytes (Shiojiri N et al Cancer Research 1991; 51(10): 2611-2620, Germain L, et al Cancer Research 1988; 48: 4909-4918, Fausto N, et al Society for experimental Biology and Medicine 1993; 204: 237-241). Hepatoblasts cultured with dexamethasone (dex), DMSO, sodium butyrate or on Matrigel express markers of hepatocyte or bile duct cell differentiation (Germain L, et al Cancer Research 1988; 48: 4909-4918, Blouin M J, et al Experimental Cell Research 1995; 217(1): 22-30, Rogler L E. American Journal of Pathology 1997; 150(2): 591-602). Bile duct epithelial cells contain CKs 7, 8, 18 and 19, and γ-glutamyl transpeptidase (GGT) activity (Shiojiri N, et al Cancer Research 1991; 51(10): 2611-2620, Shiojiri N. Microscopy Research and Technique 1997; 39: 328-335).

Hepatoblasts have been isolated from primary cultures of mouse or rat embryos and their capacity to differentiate has been shown by modifying the culture substrate or the culture medium with various growth/differentiating factors (Blouin et al., 2003, Experimental Cell Research 217: 22-30) (DiPersio et al., 1991, Mol Cell Biol. 11: 4405-4414) (Gualdi et al., 1996, Genes and Development 10: 1670-1682) (Kamiya et al., 2002, Hepatology. 35: 1351-1359).

The embryonic hepatoblast resembles the adult oval cell, in that both cell types are bipotential, able to differentiate as hepatocytes or cholangiocytes. Oval cell proliferation is induced during liver regeneration if endogenous hepatocyte proliferation has been inhibited (Michalopoulos and DeFrances, 1997, Science 276: 60-66) (Alison, 1998, Current Opinion in cell biology 10:710-715) (Fausto and Campbell, 2003, Mechanisms of Development). The origin of oval cells remains a subject of debate, as is its possible filliation with hepatoblasts.

The derivation of epithelial cell lines from normal adult liver can be traced back to the primary cloning method of Coon (Coon H G. Journal of Cell Biology 1968; 39: 29A). This work was followed up by Grisham and his co-workers (Grisham J W. Annals of the New York Academy of Sciences 1980; 349: 128-137, Tsao M S, et al Experimental Cell Research 1984; 154: 38-52), who determined that a simple epithelial cell line isolated from an adult rat displayed bipotentiality in vivo (Tsao M S and Grisham J W. American Journal of Pathology 1987; 127: 168-181, Coleman W B, et al American Journal of Pathology 1997; 151(2): 353-359, and (Coleman W B and Grisham J W: Epithelial stem-like cells of the rodent liver. In: Strain A J and Diehl A M eds. Liver Growth and Repair. London: Chapman & Hall, 1998; 50-99 and references therein)).

Transgenic mice modified to inactivate or over-express key genes for growth regulation in the liver have been used to isolate hepatocyte cell lines from adult liver (Antoine B, et al Experimental Cell Research 1992; 200(1): 175-185, Wu J C, et al Proc Natl Acad Sci USA 1994; 91: 674-678, Soriano H E, et al Hepatology 1998; 27(2): 392-401). Readily accessible sources of hepatoblasts would be useful for elucidation of the molecular signals required for specification, growth, and differentiation of hepatoblasts, hepatocytes and cholangiocytes. primary cultures of hepatoblasts can be maintained in culture for only a limited time and the cells rapidly lose their differentiated properties.

To overcome this problem, it would be useful to have hepatoblast cultures. Rogler was able to isolate one bipotential cell line from E9.5 liver diverticuli (Rogler L E. American Journal of Pathology 1997; 150(2): 591-602). As an alternative approach, for surface markers that will permit identification of clonogenic hepatoblasts has recently met with success (Kubota H and Reid L M. Proc Natl Acad Sci USA 2000; 97: 12132-12137, Suzuki A, et al Hepatology 2000; 32(6): 1230-1239, Suzuki A, et al Journal of Cell Biology 2002; 156(1): 173-184). Hepatic cell lines have been established from embryos of transgenic mice (Fiorino et al., 1998, In Vitro Cell. Dev. Biol. 34: 247-258) (Amicone et al., 1997, EMBO J. 16: 495-503). Among these, MMH cell lines were shown to be non-transformed and to harbor bipotential palmate cells (Spagnoli et al., 1998, Journal of Cell Biology 143: 1101-1112). Non-transformed MMH (Met Murine Hepatocyte) lines, derived from E14 transgenic mouse embryos expressing a constitutively active form of human Met in the liver (cyto-Met), harbor bi-potential hepatic palmate cells (Amicone L, et al EMBO J 1997; 16(3): 495-503, Spagnoli F M, et al Journal of Cell Biology 1998; 143(4): 1101-1112). Palmate cells cultured in acidic fibroblast growth factor or dimethyl sulfoxide differentiate to express hepatocyte genes, whereas cultured in Matrigel they form tubular structures similar to bile ducts.

In view of the above, there remains a need to establish a simple and reproducible method to isolate hepatic cell lines that exhibit the properties of stem cells, and to investigate hepatic cell lineage relationships.

### SUMMARY OF THE INVENTION

The inventors have reported for the first time a reproducible method to isolate bipotential hepatic cell lines, which are non-transformed and immortalized without intervention of a transgene. This surprising discovery results from a prolonged period of culturing (approximately 5-16 weeks), which exceeds the limits previously reported. The cell lines can be obtained that are able to participate in adult liver regeneration, differentiate in vivo as hepatocytes and bile duct cells, and thus show the potential of true stem cells. Among the possible applications, the cells could be used as vectors to deliver drugs in the case of liver injury, or inherited diseases affecting liver function, or when a cell capable of secretion into the blood stream is required for product/drug delivery.

### BRIEF DESCRIPTION OF THE FIGURES

A more complete appreciation of the invention and many of the attendant advantages thereof will be readily obtained as the same becomes better understood by reference to the following detailed description when considered in connection with the accompanying figures, wherein:

FIG. 1. Morphology of monolayer cultures. A. subconfluent and B. confluent cultures of 9A1 cells. C. 10B1, D. 14B3, and E. 10A3 cells. Cells at low density display cytoplasmic projections, and are polygonal at confluence. However, 10A3 cells display no cytoplasmic projections and grow as epithelial islands with smooth borders. Scale bar 40 μm.

FIG. 2. Northern blot and RT-PCR analysis of mixed morphology and epithelial cell lines in basal culture conditions.

FIG. 3. Immunofluorescence analysis for cytokeratins 7, 18 and 19. A. Adult mouse liver sections show bile duct specific expression of CK7 and CK19, whereas CK18 is expressed throughout the hepatic plate. B. Cell lines 9A1, 10B1 and 14B3 homogenously express CK18 and 19. CK7 expression is present in all cells of lines 9A1 and 10B1, but not in all cells of line 14B3. The phase contrast image is of the same field shown for anti-CK7. A. CK18 scale bar 20 μm, CK7 and CK19 scale bar 10 μm. B. scale bar 20 μm.

FIG. 4. Differentiation protocols used for cell cultures. A. 5 day aggregates of 9A1 cells displaying an outer layer of cuboidal epithelium. B. 8 day Matrigel culture of 14B3 showing an island of small dark precursor cells (left) that will later form a bile duct unit (right). C. 10 day Matrigel culture of 14B3 displaying two bile duct units. B. and C. are different areas of the same culture. Scale bar 50 μm.

FIG. 5. RT-PCR analysis of hepatic cells cultured as aggregates for 5 days shows up-regulation or induction of hepatocyte gene functions, and in some instances down-regulation of bile duct/oval cell markers. The H₂O control is a negative control. −: basal culture conditions, Agg: aggregates cultured for 5 days. HPRT: internal loading control.

FIG. 6. Cells cultured in Matrigel for 10 days express bile duct/oval markers as shown by RT-PCR analysis. HPRT: internal loading control.

FIG. 7. Down-regulation of bile duct/oval cell markers when cells are replated after Matrigel culture. Matrigel: Cells cultured in matrigel 10 days. Replated: cells cultured in matrigel 10 days, replated on collagen coated dishes and cultured 5 days.

FIG. 8. Re-expression of bile duct/oval cell markers that had been repressed by culture of cells as aggregates, and extinction of hepatocyte markers that had been induced by aggregation. Agg: cells cultured as aggregates 5 days. Replated: cells cultured as aggregates 5 days, replated on collagen coated dishes and cultured 5 or 10 days.

FIG. 9 Mouse Alb-uPa liver 3 weeks after injection of BMEL cell line 9A1-GFP. Immunohistochemistry staining (brown) showing GFP expressing cells contributing to the liver as hepatocytes (A, B) or as bile ducts (C, d). The purple stain (A) corresponds to necrotic areas. Magnification: A 100×, B 200×, C and D 400×.

FIG. 10 Mouse Alb-uPa liver 3 weeks after injection of BMEL cell line 9A1-GFP. Immunohistochemistry staining (brown) on adjacent serial sections showing a bile duct formed by GFP positive cells which express the bile duct specific marker CK19. Magnification 400×.

FIG. 11 Mouse Alb-uPa liver 3 weeks after injection of BMEL cell line 9A1-GFP. Immunohistochemistry staining (brown) revealing Albumin, GFP, or CK 19 expression on adjacent serial sections. The hepatocytes which express GFP also express albumin, the bile ducts formed by GFP expressing cells also express CK19. Magnification 400×.

FIG. 12 Mouse Alb-uPa liver 3 weeks after injection of BMEL cell line 14B3-GFP. Immunohistochemistry staining (brown) revealing GFP or DPPIV expressing cells on adjacent serial sections. The GFP expressing cells also express the hepatocyte marker DPPIV. Magnification 200×.

FIG. 13 Mouse Alb-uPa liver 5 weeks after injection of BMEL cell line 9A1-GFP. Immunohistochemistry staining (brown) revealing GFP expressing cells contributing to the parenchyma. Magnification 100×.

FIG. 14 Mouse Alb-uPa liver 5 weeks after injection of BMEL cell line 14B3-GFP. Immunohistochemistry staining (brown) revealing the presence of MHC class I haplotype H2K positive cells of BMEL origin. Magnification 200×.

FIG. 15 Mouse Alb-uPa liver 5 weeks after injection of BMEL cell line 14B3-GFP. Immunohistochemistry staining (brown) on adjacent serial sections revealing cells which express DPPIV, GFP, or CK19. Areas of GFP positive cells are encircled. The GFP expressing cells that have differentiated to hepatocytes express DPPIV, whereas the GFP expressing cells which have differentiated into bile ducts express CK19. Magnification 100×.

FIG. 16 Mouse Alb-uPa liver 8 weeks after injection of BMEL cell line 9A1-GFP. Immunohistochemistry staining (brown) revealing DPPIV, GFP, or CK19 expression on adjacent serial sections. Cells which express GFP have differentiated as hepatocytes which express DPPIV and not the bile duct specific marker CK19. Magnification 200×.

### DETAILED DESCRIPTION OF THE PREFERRED EMBODIMENTS

As used herein, ADH is alcohol dehydrogenase, AFP is α-fetoprotein, Apo is Apolipoprotein, BMEL is bipotential mouse embryonic liver, CK is cytokeratin, Cx 43 is connexin 43, Dex is dexamethasone, GGT is γ-glutamyl transpeptidase, HNF is hepatocyte nuclear factor, IB4 is integrin beta 4, and LETF is liver-enriched transcription factor.

Hepatic Cells

It has been widely observed that isolation of hepatic cell lines from mice is aleatory (Wu J C, et al. Proc Natl Acad Sci USA 1994; 91: 674-678, Wu J C, et al Cancer Research 1994; 54(22): 5964-5973, Fiorino A S, et al In Vitro Cell Dev Biol 1998; 34: 247-258). The present invention demonstrates that hepatic cell lines can be reproducibly derived from E14 embryos of multiple mouse strains.

The above-mentioned MMH cell lines were isolated from transgenic cyto-Met mice embryos. To verify the role of the cyto-Met transgene, the Inventors isolated similar cell lines from non-transgenic mouse embryos. Using the culturing procedure disclosed herein, colonies of cells developed in the plates originating from transgenic embryos after approximately 4 weeks, and from non-transgenic embryos after about 8 weeks. The experiment was repeated with non-transgenic mouse embryos from numerous genetic backgrounds and colonies of cells giving rise to cell lines developed between 5 to 16 weeks.

Beginning with embryos originating from a cross between CBA/J and C57Bl/6J mice, 16 cell lines were established and characterized.

The properties of BMEL (Bipotential mouse embryonic liver) lines and their subclones, as well as the Inventors' earlier experience with MMH cells, permit the Inventors to define the hallmarks of bipotential lines (Spagnoli F M, et al Journal of Cell Biology 1998; 143(4): 1101-1112, Spagnoli F M, et al Journal of Cell Science 2000; 113: 3639-3647). First, they show a mixed morphology, containing both palmate-like cells and epithelial cells. Second, they present an uncoupled phenotype, expressing LETFs but not hepatocyte functions, although these functions are inducible.

Northern blot and RT-PCR analysis showed that cells of these lines all express the liver-enriched transcription factors (LETF) HNF1α, HNF1β, HNF3α, β, γ, HNF4α, GATA4 and some express the liver functions albumin (Alb) and apolipoprotein (Apo) B. Three cell lines that expressed the LETF without expressing the liver functions Alb and Apo B were studied further to determine whether the expression of these functions could be induced. Indeed, the expression of these genes and others characteristic of hepatocytes (Apo AIV, Aldolase B, Alcohol dehydrogenase) is induced by culture of the cells in dexamethasone, or as aggregates. To determine whether these cells could differentiate as cholangiocytes, they were cultured in Matrigel. The cells formed elaborate networks within which structures similar to bile duct units developed. Gene expression analysis showed that the cells expressed bile duct epithelial cell markers such as HNF6, γglutamyl transpeptidase IV, c-kit, and Thy-1.

The BMEL cell lines can be isolated from non-transgenic mouse embryos of many different genetic backgrounds. In a similar manner, the cell lines could be obtained from adult mouse liver tissues. In addition, it may be possible to apply the same technology to embryonic and adult tissues from other mammals, including, for example, human, bovine, porcine, equine, feline, canine, etc. In one embodiment, the cells are obtained from embryonic liver tissue, preferably about 14 dpc mouse embryonic liver tissue.

The cell lines have been cloned and the clones present the same characteristics and bipotentiality as the parental cells. Furthermore, the BMEL cells are immortalized but non-transformed, they do not grow in soft agar and do not form tumors in nude mice after subcutaneous injection. Two examples of the BMEL cell lines are BMEL-14B3 and BMEL 9A1 deposited at the Collection Nationale de Cultures de Microorganismes, CNCM on Oct. 3, 2003 under the accession numbers I-3100 and I-3099, respectively.

These stem cells can be frozen and thawed, maintained in culture in basal medium when they are non-differentiated, and induced to differentiate at will.

As used herein, “differentiated” as it relates to the cells of the present invention means that the cells have developed to a point where they are programmed to develop into a specific type of cell and/or lineage of cells. Similarly, “non-differentiated” or “undifferentiated” as it relates to the cells of the present invention means that the cells are stem or progenitor cells, which are cells that have the capacity to develop into various types of cells within a specified lineage, e.g., hepatic lineage.

As used herein, the terms “wild-type mouse” or “non-transgenic mouse” when they refer to the origin of the cells of the present invention means that the genome of said mouse does not comprise any transgene liable to immortalize said cells of the invention. However, other genetic modifications of the mouse genome should not influence the potential to isolate the cell lines of the invention. In a similar manner, “wild-type” or “non-transgenic” has the same definition when referencing other mammals as well.

The definition of a stem cell includes the following principles: 1) the cells are non-differentiated in basal culture conditions where they undergo self-renewal 2) the cells can differentiate along at least two pathways 3) the cells are not transformed 4) the cells can differentiate in vivo and participate in tissue formation. The BMEL cells of the present invention fulfill all of these criteria.

Culturing Procedure/Conditions

One embodiment of the culturing procedure is a variant of Coon's method of primary cloning (Coon H G, et al Journal of Cell Biology 1968; 39: 29A).

The culture media used to prepare the cells described herein can be any known physiologically acceptable liquid medium. The culture medium contains various organic and inorganic components that support cell proliferation and may contain various conventional medium components, for example, MEM, DMEM, RPMI 1640, Alpha medium, McCoy's medium, and others. In a particular embodiment, the culture medium is RPMI 1640 or William's medium.

The cultures could be supplemented with serum, such as those obtained from calf, fetal calf, bovine, horse, human, newborn calf. In a particular embodiment, the culture medium was supplemented with fetal calf serum. The serum may be present in the culture in an amount of at least 1% (v/v) to 50% (v/v), preferably the serum concentration is from 5 to 25% (v/v). In an alternative embodiment, the serum could be replaced in whole or in part with one or more serum replacement compositions, which are known in the art.

The cultures may also contain one or more cytokines, growth factors, or growth inhibitors which could affect the differentiation pathway of the cells and the like conventionally used in cell culture. For example, epidermal growth factor may be used. The cultures are generally maintained at a pH that approximates physiological conditions, e.g., 6.8 to 7.4 and cultured under temperatures of about 37° C. and under a carbon dioxide containing atmosphere, e.g., at least 5%, at least 7%, at least 10% etc.

The density of cells in the culture may vary widely and will dependent on the viability of the cells after initial introduction into the culture system. In one embodiment, the cells are plated and maintained at a cell density of about 5×10³ to about 3×10⁵ cells/cm² in culture medium.

The time for culturing will vary depending on the source of mammalian cells as well as the specific culture conditions. However, in a preferred embodiment, the culturing procedure is carried out for at least about little more than 1 month (35 days) to 4 months (120 days) or longer, if desired. In a particular embodiment, the cells are cultured for at least about 2 months. In a particular embodiment, the cells are cultured for at least about 3 months. During this period of time the medium is replaced with fresh medium periodically, or alternatively continuously. For example, the medium can be replaced every 3 to 7 days during the culturing period.

Therapeutic Applications

To affirm that the cells of the invention can become functional adult hepatocytes, reimplantation in vivo can be tested. Several mouse liver repopulation models exist, including the albumin-urokinase-type plasminogen activator (Alb-uPa) transgenic mouse and fumarylacetoacetate hydrolase deficient (FAH−/−) mouse (Shafritz D A and Dabeva M D. Journal of Hepatology 2002; 36: 552-564 and references therein).

The inventors have shown that the BMEL cells injected into the spleen of a diseased mouse liver are able to home, engraft, proliferate, differentiate as hepatocytes or bile duct cells, and thus participate in liver regeneration. The engraftment is stable since the cells are present 8 weeks after infusion.

The cells of the present invention can also be used to generate hepatocytes and/or bile ducts in vitro. The cells of the present invention are then cultured under specific conditions suitable to induce the differentiation into hepatocytes or bile ducts. Examples of such conditions are given in the Examples.

The cells could be induced to differentiate by culturing them on or in dishes coated with various substances such as Matrigel, Collagen, Laminin or Fibronectin.

The cells could perhaps be induced to differentiate into other cell types distinct from hepatocytes or bile duct cells, such as those found for example in the pancreas or intestine.

Thus, after culturing cells of the present invention could also be used to generate specific liver tissue in vitro or in vivo. Normal liver tissue comprises both the hepatocytes as well as the bile ducts and in addition mesenchymal cell types. In generating liver tissue, the hepatic cells of the present invention are cultured under specific conditions suitable to induce the differentiation into hepatocytes and bile ducts. The formation of liver tissue could require the addition of mesenchymal cells. In an alternative embodiment, the hepatic cells of the present invention could be directly infused into the mammal whereby the cells home into the proper location in the body and generate/regenerate liver tissue.

This generation of hepatocytes, bile ducts, and/or liver tissue should be useful to treat or provide a therapeutic benefit to an individual suffering from a liver injury caused by physical or genetic etiologies as well as treating individuals with inheritable liver diseases.

The BMEL cell lines are transduced efficiently with the TRIP-GFP lentiviral vector and the cells express the protein of interest. Therefore, other vectors well known in the art as well as other genes of interest, such as, for example, genes for therapeutic purposes, can also be introduced into the cells. As a result one embodiment of the present invention is the transduction of the cells described herein with polynucleotides encoding one or more proteins capable of providing a therapeutic benefit to the individual receiving the cells and/or improving, altering, or changing the physiology of the cells to facilitate the formation of liver tissues and/or improve liver function. In one embodiment, the cells can further be transduced with a suicide gene. In the event where cells are injected into the liver would need to be removed, the suicide gene expression would permit the selective elimination of the injected cells. In still another embodiment, the transduced cells could be injected into a mammal.

Investigational Applications

While much is known of the events that lead to hepatocyte differentiation, the initiator genes for bile duct differentiation have not yet been identified. BMEL cells or other cells of the invention could be exploited to define genes that are essential for bile duct differentiation and morphogenesis. The Inventors observed that 9A1 cells express bile duct markers without forming bile duct units, whereas 10B1 and 14B3 cells express both the markers and undergo morphogenesis, implying that bile duct formation is a step-wise process, such that specification and tissue-specific gene activation occur prior to morphogenesis, similarly to the sequential events leading to hepatocyte induction and differentiation (Zaret K S. Current Opinion in Genetics and Development 2001; 11(5): 568-574). Mouse knock-out models or other mammalian models could be used to isolate bipotential hepatic cell lines in which the role of a specific gene in either hepatocyte or bile duct cell differentiation programs can be precisely defined (Hayhurst G P, et al Molecular and Cellular Biology 2001; 21(4): 1393-1403, Clotman F, et al Development 2002; 129: 1819-1828, Coffinier C, et al Development 2002; 129: 1829-1838).

The existence of bipotential hepatic lines coupled with definition of the culture conditions that induce their differentiation will make it possible to define whether hepatic cells undergo commitment to a limited differentiation potential, and if so, to identify the molecular corollaries of cell commitment. Alternatively, differentiation plasticity could prove to be the mode of regulation within the endodermal hepatic compartment. The reversibility of differentiation of BMEL cells could be related to immaturity of the cells: indeed, they do not express adult hepatocyte functions. However, the combination of specific gene induction and morphogenesis strongly suggests that differentiation has indeed occurred.

The cells of the invention, the transduced cells of the invention, or mammals in which the cells have been infused could also be used as screening tools. Accordingly, one object of the invention is a method for screening molecules which alter the normal development of the cells of the invention. The normal development of the cells of the invention depends on the culture conditions or the conditions of infusion in a mammal. For example, the normal development of a cell of the invention is the differentiation of this cell in bile duct when said cell is cultured in Matrigel. Likewise, the normal development of a cell is the differentiation of the cells into hepatocytes, for example, as described in the Examples (Culture as aggregates induced hepatocytes functions and down-regulates some oval/bile duct markers).

Another example of development is the cells remaining in a non-differentiated state when they are maintained in a basal medium.

The method of screening comprises the steps of bringing the molecule to be tested into contact with the cells of the invention under conditions for a given development of said cells and of detecting an alteration or an absence of alteration of the development of said cells. Various effects of a molecule could be tested depending on the culture or environmental conditions in which the observed cells are. For example, said method could be used to test the toxicity of a drug which could alter the development of an embryo's liver when administered to pregnant females or to test the ability of some molecules to promote liver regeneration, etc.

In another embodiment of the invention, non-human mammals infused with the non-transduced or transduced cells of the invention are also an object of the invention.

### EXAMPLES

### Example 1

### Hepatic Cell Line Isolation

Each liver at 14 dpc was separately dissected in PBS, homogenized in an Elvehjem Potter in Hepatocyte attachment medium (Invitrogen, Groningen, The Netherlands) containing 10% fetal calf serum (FCS) and antibiotics, inoculated into two 100 mm petri dishes and cultured overnight. The next day, and weekly thereafter, the medium was replaced with RPMI 1640 (Invitrogen) containing 10% FCS, 50 ng/ml EGF, 30 ng/ml IGF II (PeproTech, Rocky Hill, USA), 10 μg/ml insulin (Roche, Mannheim, Germany) and antibiotics. Cell lines were obtained from scraped colonies inoculated into 12 well microtiter plates. Cells were dissociated with trypsin-EDTA and passaged every 3 days, corresponding to 4-5 generations. Cells were cultured on Collagen I (Sigma, St. Louis, USA) coated dishes in a humidified atmosphere with 7% CO₂ at 37° C.

Soft Agar and Tumor Formation Assays, and Karyotype Determination

For the soft agar assay, 1×10⁴ or 1×10⁵ cells were inoculated as detailed in (Spagnoli et al Journal of Cell Biology 1998; 143(4): 1101-1112), using BW1J cells as positive control. For the tumor formation assay, 6 week old male Balb/c nu/nu mice were injected subcutaneously with 1×10⁵ or 1×10⁶ cells for each cell line tested. Two mice were tested for each cell concentration. Mice were inspected twice weekly for tumors during 7 weeks and microscopically after sacrifice. Karyotype analysis was performed as described in (Spagnoli F M, et al Journal of Cell Biology 1998; 143(4): 1101-1112) on cells at passages 6 and 12; superimposable results were obtained and cumulative data are presented.

Cell Aggregation, Culture in Matrigel, and Replating

1) Aggregates: 5×10⁶ cells were inoculated onto a 100 mm bacteriological grade petri dish to which cells do not attach, but form floating aggregates within 24 hours. Aggregates were collected for RNA extraction 5 days after inoculation. 2) Matrigel: 0.5 ml of Matrigel (Becton Dickinson, Bedford, USA) was placed onto 60 mm petri dishes, permitted to set for 1 hour, and 0.5×10⁶ cells were plated in culture medium supplemented with 100 ng/ml HGF (R&D Systems, Oxon, UK). Cells were recovered after 10 days by 2 hours Dispase (Becton Dickinson) digestion at 37° C. prior to RNA extraction. 3) Replating: Aggregates in culture for 5 days were placed on collagen coated dishes to which they attached and RNA was extracted 5 or 10 days after without passaging. Cells in Matrigel culture for 10 days were dissociated by Dispase digestion (20 min) and replated on collagen coated dishes, and RNA was extracted 5 days later. 4) To define optimal induction conditions, cells were cultured on gelatin-coated dishes (0.1% in PBS) or with 100 ng/ml HGF (R&D Systems), 100 ng/ml aFGF (Invitrogen) combined with 10 μg/ml Heparin (Invitrogen), or 10⁻⁶M dexamethasone (dex) (Sigma), or without serum, each for 5 days, or 10⁻⁶M dex for 48 hrs including 10⁻⁴M 8-(4-chlorophenylthio)-cAMP (cAMP) (Sigma) for the last 24 hrs, prior to RNA extraction.

Immunofluorescence Analysis

Indirect immunofluorescence on cryostat sections of adult mouse liver and on monolayer cultures was performed as described (Spagnoli F M, et al Journal of Cell Biology 1998; 143(4): 1101-1112). The primary antibodies were rat monoclonal anti-CK18 and anti-CK19 (TROMA 2 and 3) a gift from R. Kemler (Max-Planck Institute of Immunobiology, Freiburg, Germany) and mouse monoclonal anti-CK7 (Progen, Heidelberg, Germany). The secondary antibody was rabbit anti-rat IgG conjugated to FITC (Sigma) and goat anti-mouse IgG conjugated to FITC (Caltag, Hamburg, Germany).

RNA Analysis

Total cellular RNA was extracted from cells according to standard protocols. Northern blots and ³²P-labeled cDNA inserts were prepared as described in (Chaya D, et al Molecular and Cellular Biology 1997; 17(11): 6311-6320, Spagnoli F M, et al Journal of Cell Biology 1998; 143(4): 1101-1112). Reverse transcription was performed using 5 μg of total RNA with random hexamers and SuperScript II reverse transcriptase (Invitrogen) according to manufacturer's protocols. The PCR conditions were 95° C. 5 min; 95° C. 30 sec, annealing temperature 30 sec, and 72° C. 30 sec, 28 to 34 cycles; 72° C. 10 min. After RT-PCR, DNA fragments were resolved on 1.5% agarose gels. Forward and reverse primers used for specific amplification can be found in these references or obtained from the authors: HNF6 (Lemaigre F P, et al Proc Natl Acad Sci USA 1996; 93(18): 9460-9464), albumin (Li J, et al Genes and Development 2000; 14: 464-474), c-Kit (accession #D12524), Thy-1 (accession #M10246), Cx 43 (accession #M63801), CD 34 (accession #S69293), Aldolase B (accession #M10149), GGT IV (Holic N, et al American Journal of Pathology 2000; 157(2): 537-548), ADH (accession #M11307), PAH (Li J, et al Genes and Development 2000; 14: 464-474), PEPCK (accession #AF009605), TAT (accession #M18340), IB4 (Couvelard A, et al Hepatology 1998; 27(3): 839-847), HPRT (Li J, et al Genes and Development 2000; 14: 464-474), AFP (Li J, et al Genes and Development 2000; 14: 464-474), TFN (Li J, et al Genes and Development 2000; 14: 464-474), Apo AIV (Li J, et al Genes and Development 2000; 14: 464-474), Apo B (Li J, et al Genes and Development 2000; 14: 464-474), HNF3α (Li J, et al Genes and Development 2000; 14: 464-474), HNF3β (Li J, et al Genes and Development 2000; 14: 464-474).

Cloning of Embryonic Hepatic Cell Lines

500 cells, from suspensions assessed by microscopic examination to consist of mainly single cells, from each cell line were plated on mitomycin C arrested mouse embryonic fibroblast feeders or on collagen coated 100 mm dishes for subcloning. 2 weeks later isolated colonies were scraped, plated onto collagen coated 12-well microtiter plates and expanded.

Results

Hepatic Cell Lines Isolated from Mice of Many Genetic Backgrounds.

To determine whether the protocol that permits isolation of bipotential hepatic lines from cyto-Met transgenic mice can also be successful with wild-type mice, we tested four different genetic backgrounds (Table I). Embryos at E14 were dissected individually and dissociated cells from each liver were plated. Primary cultures began to degenerate after 2 weeks of plating, yet some live cells remained. As controls, homozygous cyto-Met embryonic livers were used and within 4 weeks, as expected (Amicone L, et al Embo 1997; 16(3): 495-503), colonies of proliferating epithelial cells were observed in the dishes. Importantly, similar colonies of healthy looking cells were present 5 to 12 weeks after plating in about one third of the cultures from wild-type mice, a frequency comparable to that obtained with homozygous cyto-Met mice (Table I). Hepatic cell lines were isolated from different genotypes with varying efficiencies: the most favorable backgrounds were CBA/J and DBA/J and the least favorable were BALB/c and C57BL/6J. Once islands of epithelial cells growing in cobblestone fashion appeared, they grew vigorously and were picked. The cells are thereafter passaged at low density (8.6×10³ cells/cm²) every 4 days and in some cases for over 60 cell generations.

TABLE I
Hepatic cell lines can be derived from
mice of multiple genetic backgrounds.

| | % of livers giving cell | | Plate and Wait | | |
|---|---|---|---|---|---|
| | lines | | time | | # Cell |
| | (number of embryos | | for colony | | lines |
| Cross | tested) | | emergence | | frozen |
| cyto-Met × cyto-Met | 29.4 | (17) | 4 | w | 15 |
| CBA/J × CBA/J | 100 | (3) | 7-16 | w | 7 |
| DBA/2J × DBA/2J | 57 | (7) | 5-9 | w | 2 |
| DBA/2J × BALB/c | 37.5 | (8) | 13 | w | 3 |
| CBA/J × C57BL/6J | 37.5 | (16) | 5-8 | w | 16 |
| CBA/J × DBA/2J | 25 | (8) | 6-12 | w | 10 |
| C57BL/6J × | 20 | (5) | 11 | w | 1 |
| BALB/c | | | | | |
| C57BL/6J × | 6.6 | (15) | 8 | w | 1 |
| C57BL/6J | | | | | |
| C57BL/6J × DBA/2J | 0 | (11) | | | |

Mean time between picking the colony to freezing: 2 weeks, range 2-5 weeks.
Cellular doubling time: 24-30 hours.

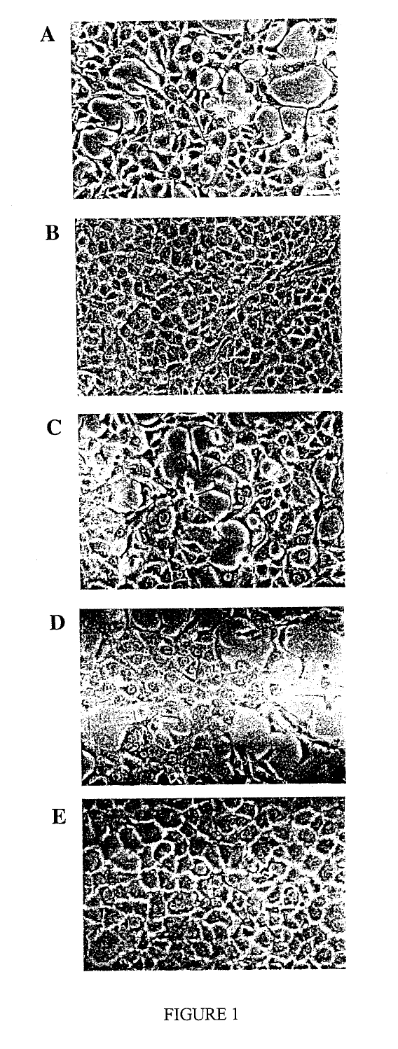

Of sixteen cell lines isolated from CBA/Jx C57Bl/6J mice (Table II), eleven displayed a mixed morphology composed of palmate-like cells with cytoplasmic projections and epithelial cells with granular cytoplasm and polygonal form (FIG. 1A, C, D), while five were composed only of epithelial cells (FIG. 1E). The photographs reveal a smooth transition from palmate-like to epithelial cells, as though the cytoplasmic projections could appear on any cell not surrounded by neighbors. In contrast, at high density the cultures appear epithelial (FIG. 1B).

To determine whether the cells are anchorage dependent, they were plated in soft agar. All failed to grow after 3 weeks, except three cell lines that formed organized tubule-like structures. These were tested in nude mice and no tumors were observed (Amicone L. and Tripodi M., personal communication). The Inventors conclude that these cells are not transformed.

TABLE II
Isolation of hepatic cell lines from wild-type
CBA/J/C57BL/6J mouse embryos

| | | | Embryonic |
|---|---|---|---|
| Embryo # | # of Colonies | LETF | Liver functions |
| 1 | 3 | + | + |
| 9 | 1 | + | − |
| 10 | 1 | + | + |
| | 1 | + | − |
| 12 | 2 | + | + |
| 14 | 6 | + | + |
| | 1 | + | − |
| 16 | 1 | + | − |

LETF: liver-enriched transcription factors HNF1α, HNF4α and HNF3
Embryonic liver functions: AFP and transthyretin

Cells Express Liver-Enriched Transcription Factors But Hardly any Liver-Specific Functions.

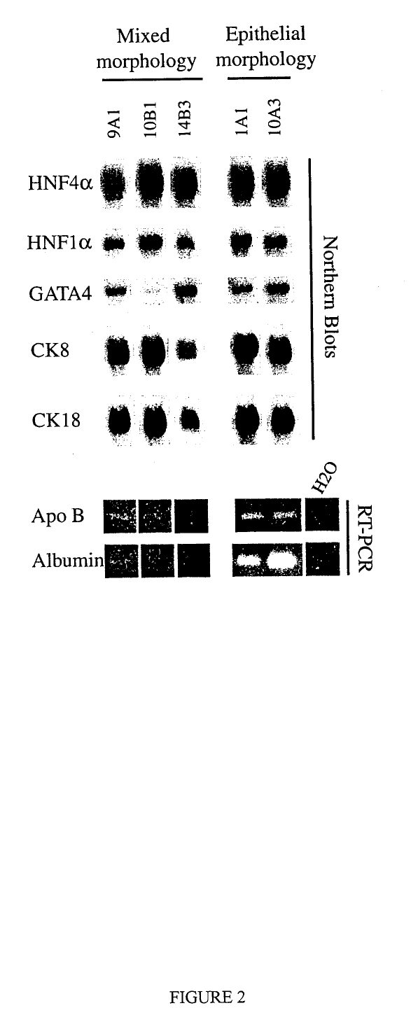

RNA from cell lines of mixed morphology (9A1, 10B1, 14B3) or purely epithelial morphology (1A, 10A3) was analyzed, all express HNF4α, HNF1α, and GATA4 as well as CK8 and 18 (FIG. 2). In addition, only cells from epithelial lines strongly express the hepatocyte markers Apo B and albumin (FIG. 2). The undifferentiated phenotype of cell lines of mixed morphology is reminiscent of bipotential palmate cells.

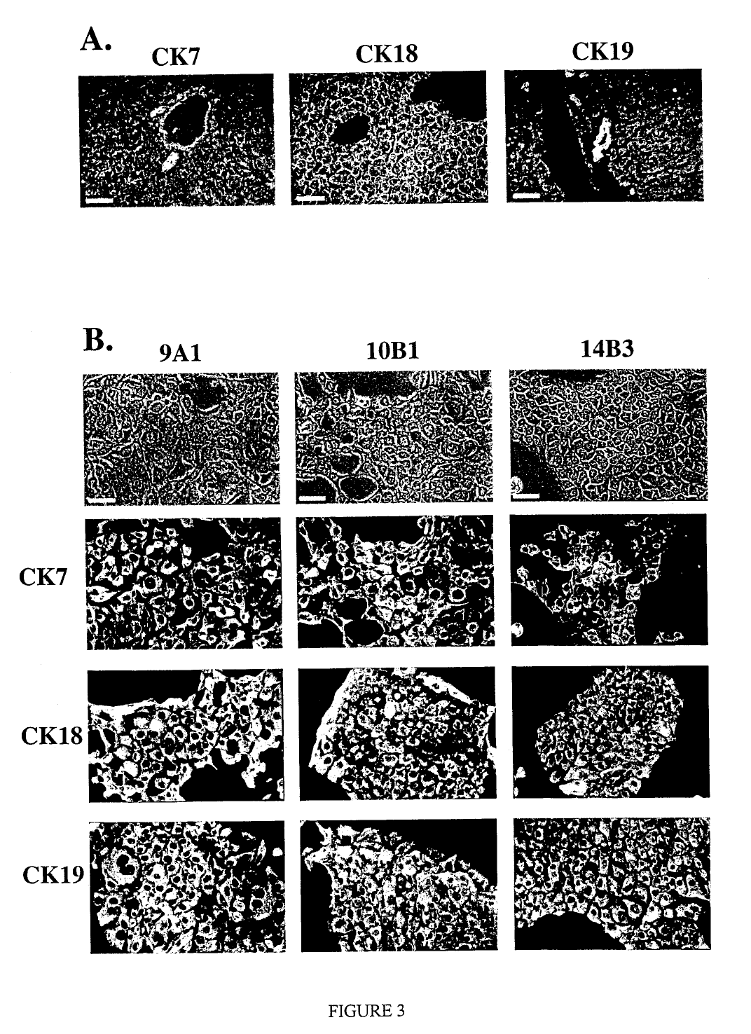

It is known that, CK18 is expressed in both hepatocytes and bile duct cells, whereas CK7 and 19 are expressed in bile duct cells (FIG. 3) (Shiojiri N, et al Cancer Research 1991; 51(10): 2611-2620, Germain L, Cancer Research 1988; 48: 4909-4918). Immunofluorescence analysis revealed that CKs 18 and 19 were expressed in all cells of the culture (FIG. 3). Unexpectedly cells of lines 9A1 and 10B1 all expressed CK7 whereas patches of cells with large clear cytoplasms of line 14B3 did not (FIG. 3).

Karyotype analysis of 9A1, 10B1, and 14B3 cells revealed bimodal karyotypes with one population at 39 chromosomes and a second with double this number (Table III). Thus, all three lines contain cells with a near diploid karyotype at passages 6 and 12, with no decrease in near-diploid metaphases with time in culture.

Culture as Aggregates Induces Hepatocyte Functions and Down-Regulates Some Oval/Bile Duct Markers.

Table IV presents the markers which have been used to determine whether cells are bipotential. Because there is overlap among markers expressed by hepatoblasts, bile duct and oval cells, individual markers are not considered diagnostic: rather, groups of markers were analyzed.

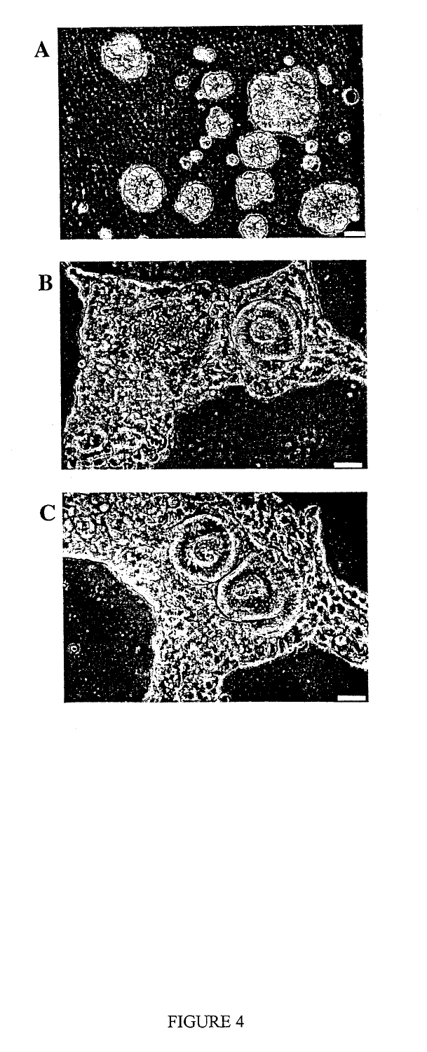

To assess whether the cells of mixed morphology could differentiate to express hepatocyte functions, they were cultured in the presence of hepatocyte growth factor (HGF), acidic fibroblast growth factor (aFGF), dex, dex+cAMP, in medium without serum, on gelatin or as aggregates (Greengard O. Science 1969; 163(870): 891-895, Landry J, et al Journal of Cell Biology 1985; 101(3): 914-923, Coleman W B, et al Journal of Cellular Physiology 1994; 161: 463-469, Lazaro C A, et al Cancer Research 1998; 58: 5514-5522, Spagnoli F M, et al Journal of Cell Biology 1998; 143(4): 1101-1112). RT-PCR analysis revealed that the most differentiated hepatocyte phenotypes were obtained after treatment with dex+cAMP or growth as aggregates. Aggregates contained tightly packed cells with an exterior surface of cuboidal epithelium and in some cases a central lumen (FIG. 4A).

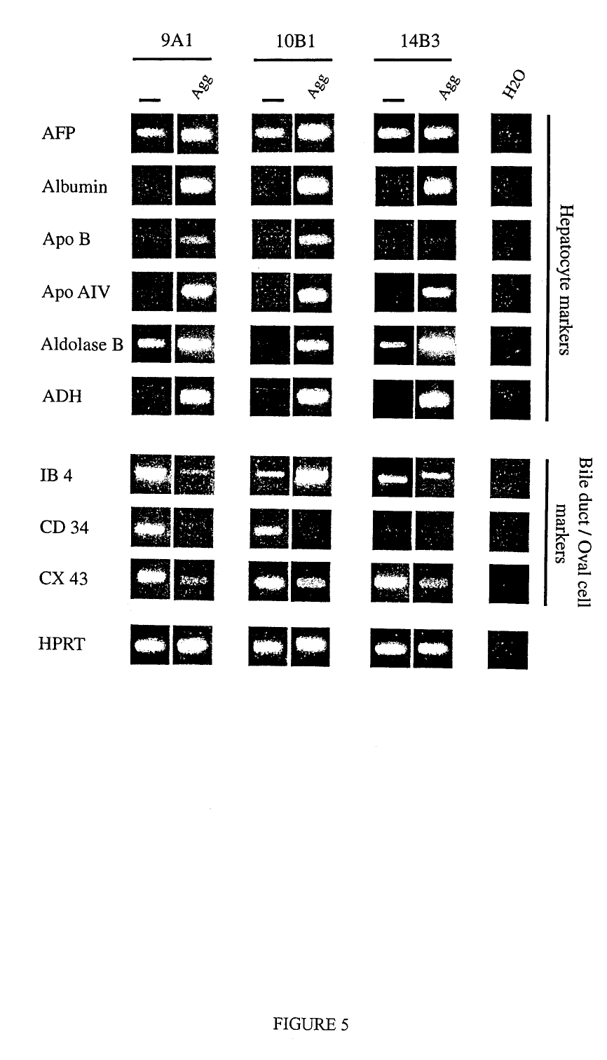

Hepatocyte markers: the upregulation of AFP and aldolase B, coupled with the induction of albumin, Apo B, Apo AIV, and ADH, indicated that the cells within aggregates had differentiated as hepatocytes (FIG. 5 and Table V). Transcripts of transferrin, CK8, 18 and 19 were present at similar levels irrespective of the culture conditions, transcription of the neonatal hepatocyte-specific genes TAT and PEPCK was not induced.

TABLE III
Karyotypes

| | |
|---|---|
| Near | |
| diploid | Hypotetraploid |

| Cell | % | | % | |
|---|---|---|---|---|
| line | Metaphases | Mode | Metaphases | Mode |
| 9A1 | 49.5 | 39 | 50.5 | 78 |
| 10B1 | 43 | 39 | 57 | 78 |
| 14B3 | 17.5 | 38 | 82.5 | 78 |

Based upon the analysis of 81 metaphases for 9A1, 76 for 10B1, and 74 for 14B3

Bile duct/Oval cell markers: many markers are in common, including GGT IV, CD 34, c-Kit, IB4, Cx 43 and CK7 and 19 (Table IV) (Holic N, et al American Journal of Pathology 2000; 157(2): 537-548, Omori N, et al Hepatology 1997; 26(3): 720-727, Fujio K, et al. Experimental Cell Research 1996; 224(2): 243-250, Zhang M and Thorgeirsson S S. Experimental Cell Research 1994; 213: 37-42). Thy-1 is expressed only in oval cells (Petersen B E, et al Hepatology 1998; 27(2): 433-445). In basal culture conditions, CD 34, c-Kit, IB4, Cx 43, and CK7 and 19, but not Thy-1 or GGT IV were expressed (FIGS. 3, 5 and 6 and Table V). Absence of Thy-1 and GGT IV expression shows that the cells are distinct from oval cells. Significantly, induction of hepatocyte markers by aggregation coincided with down-regulation of the bile duct/oval cell markers CD 34 and Cx 43 (FIG. 5), while expression of IB4 was downregulated only in 9A1 cells. Neither GGT IV nor Thy-1 transcripts were detected. These results show an impressive and essentially unidirectional induction of hepatocyte differentiation when cells are cultured as aggregates.

TABLE IV
Cell types in the liver: gene expression patterns.

| | | | Bile | | |
|---|---|---|---|---|---|
| | Hepato- | Oval | Duct | Hepato- | BMEL |
| Markers | blast | cell | cell | cyte | (Basal) |
| Oval | | | | | |
| Thy-1 | NF | + | − | − | − |
| Bile duct/Oval | | | | | |
| GGT IV | + | + | + | − | − |
| CD 34 | NF | + | + | − | + |
| c-kit | NF | + | + | − | +/− |
| IB 4 | − | + | + | − | + |
| CX 43 | NF | + | + | − | + |
| CK 7 and 19 | − | + | + | − | + |
| Bile duct/Hepatocyte | | | | | |
| HNF6 | + | NF | + | + | + |
| Hepatocyte | | | | | |
| AFP | + | + | − | + | + |
| Albumin | + | + | − | + | − |
| Apo AIV | NF | NF | − | + | − |
| ADH | NF | NF | − | + | − |
| Apo B | NF | NF | − | + | +/− |
| Aldolase B | NF | NF | − | + | + |

NF: not found in the literature −: Not expressed +/−: Trace expression +: Expressed
REFERENCES: Thy-1: (36), (50) GGT IV: (1), (36), (27), CD 34: (33), c-Kit: (33), (34), IB 4: (28), Cx 43: (35), CK 7 and 19: (1), (2), (36), HNF6: (47), AFP: (1), (5), (36), Albumin: (1), (2), (5), Apo AIV: (9), ADH: (24), Apo B: (9), Aldolase B: (24)

Culture in Matrigel Induces Morphogenesis of Bile Duct Units and Expression of Bile Duct/Oval Cell Markers.

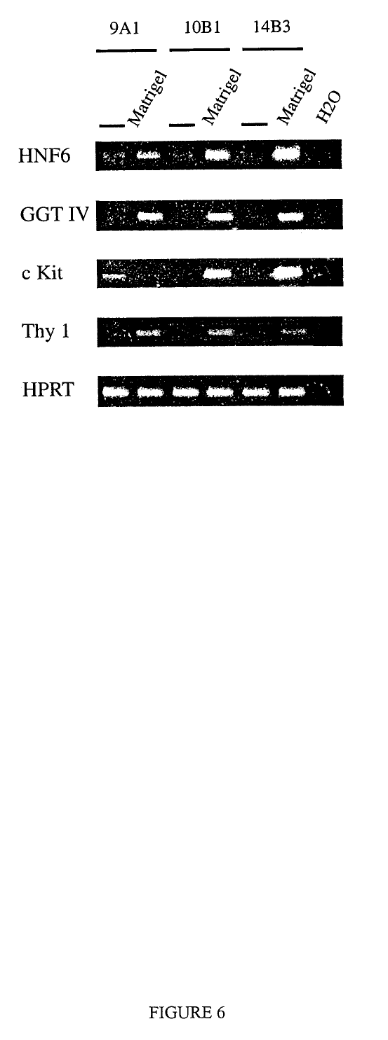

To assess whether cells of mixed morphology can differentiate into bile duct cells as well as into hepatocytes, they were cultured in Matrigel, previously shown to favor bile duct cell differentiation (Paradis K and Sharp H L. J Lab Clin Med 1989; 113: 689-694). The cells created a web throughout the dish, within which foci of small dark cells appeared after 5 days (FIG. 4B). These foci became organized and developed 1-2 days later as doughnut-like structures identical to bile duct units (Mennone A, et al Proc Natl Acad Sci USA 1995; 92: 6527-6531, Cho W K, et al Am J Physiol Gastrointest Liver Physiol 2001; 280: G241-G246) (FIGS. 4 B and C), spherical three-dimensional structures of tightly packed columnar epithelium with a central lumen. RT-PCR analysis of 10 day Matrigel cultures revealed that concomitant with morphogenesis, the bile duct/oval cell markers had been strongly induced, including HNF6, GGT IV, c-Kit and Thy-1 (FIG. 6). 14B3 and 10B1 cells displayed the most striking bile duct differentiation, with both bile duct units and robust induction of all markers examined. However, 9A1 cells did not form bile duct units, yet three of the marker genes were induced: HNF6, GGT IV and Thy-1. The induction to differentiate in Matrigel was not specific since hepatocyte markers albumin, ADH, and aldolase B were also induced (Table V).

These results show that cells of mixed morphology are non-differentiated and bipotential, able to differentiate as hepatocytes or as bile duct cells. The bipotential nature of the cells, which are now designated as BMEL (Bipotential Mouse Embryonic Liver) (Table V) was verified in cloned progeny.

Clonal Descendants of BMEL Cells Retain Mixed Morphology and Bipotentiality.

Daughter clones of the three cell lines displayed the same mixed morphology as the parental lines. All the daughter clones were analyzed under basal culture conditions, as aggregates and in Matrigel. RT-PCR analysis showed that each clone displayed the same undifferentiated phenotype and bipotentiality as its parental line, with no indication of loss of differentiation potential (Table VI). Finally, no colonies were formed in soft agar (data not shown). Thus, mixed morphology and bipotentiality are both stable and heritable states of BMEL cells.

TABLE V
BMEL cells are bipotential

| | 9A1 | | | 10B1 | | | 14B3 | | |
|---|---|---|---|---|---|---|---|---|---|
| | Matrigel | Basal | Aggregates | Matrigel | Basal | Aggregates | Matrigel | Basal | Aggregates |
| Thy-1 | + | − | − | + | − | − | + | − | − |
| GGT IV | + | − | − | + | − | − | + | − | − |
| CD 34 | + | ++ | + | + | + | − | + | + | − |
| c-kit | + | + | + | + | − | − | ++ | + | + |
| IB 4 | ++ | ++ | + | + | + | ++ | + | + | + |
| Cx 43 | ++ | ++ | + | ++ | ++ | + | ++ | ++ | + |
| HNF6 | ++ | + | ++ | ++ | + | ++ | + | − | + |
| AFP | + | + | ++ | + | + | ++ | + | + | ++ |
| Albumin | + | − | ++ | + | − | ++ | + | − | ++ |
| Apo AIV | ND | − | ++ | ND | − | ++ | ND | − | ++ |
| ADH | + | − | + | + | − | + | + | − | ++ |
| Apo B | ND | + | ++ | ND | − | ++ | ND | − | ++ |
| Aldolase B | ++ | + | ++ | ++ | − | ++ | ++ | + | ++ |

ND: not determined −: no expression +: expressed ++: strongly expressed

Reversibility of BMEL Cell Differentiation

It has previously been shown that cells of a simple epithelial line from rat undergo differentiation when transplanted in vivo. Upon re-inoculation in culture, undifferentiated cells were present, suggesting either that differentiation is reversible, or that only the undifferentiated cells retained growth potential in culture (Grisham J W, et al Proceedings of the Society of Experimental Biology and Medicine 1993; 204: 270-279). To determine whether the induction of BMEL cell differentiation by aggregation or by culture in Matrigel is irreversible, such cultures were replated as monolayers on collagen coated dishes.

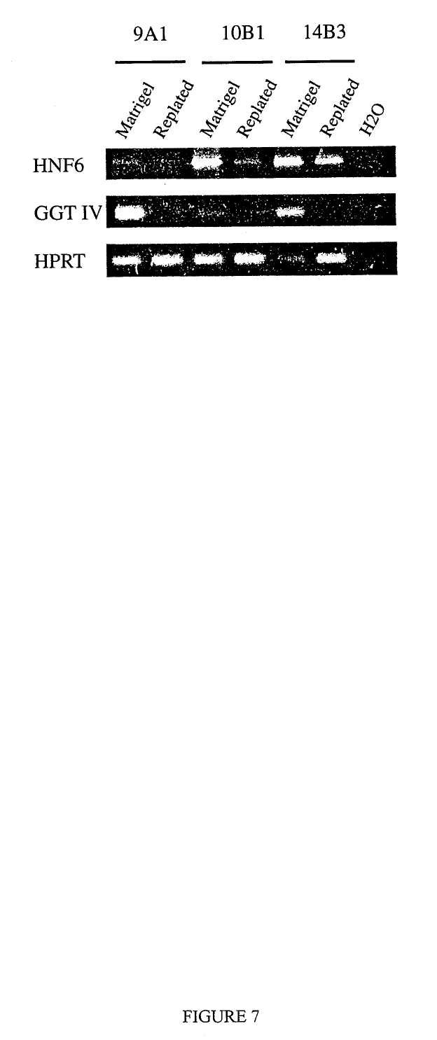

Two of the most diagnostic markers of differentiation in Matrigel culture are HNF6, upregulated in both hepatocytes and bile ducts, and GGTIV, excluded from hepatocytes and expressed in cholangiocytes and oval cells. BMEL cells induced for 10 days in Matrigel were harvested for RNA, or replated as monolayers and again harvested for RNA five days later. Both HNF6 and GGTIV were induced in Matrigel and dramatically down-regulated upon replating (FIG. 7).

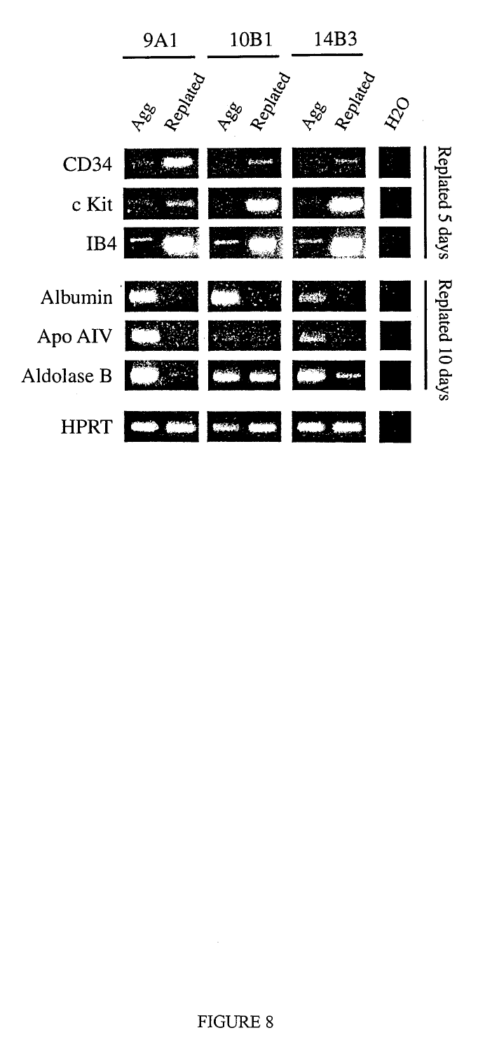

Differentiation induced by aggregation is specific for hepatocyte differentiation and several of the bile duct/oval cell markers are repressed. Cells of the three BMEL lines were grown as aggregates for 5 days and replated to grow as monolayers for 5 or 10 days. RT-PCR analysis revealed that the bile duct/oval cell markers that had been repressed by aggregation were rapidly and strongly re-expressed by 5 days after replating (FIG. 8). Conversely, the hepatocyte markers induced by aggregation were down-regulated within 10 days after replating (FIG. 8). When either Matrigel culture or aggregates were replated, all cells attached and there was no evidence of cell death, making counter-selection of the differentiated cells an unlikely hypothesis.

While BMEL cells behave like stem cells, showing the properties of self-renewal and the potential to engage in differentiation along at least two alternative pathways, they have so far shown no evidence of another characteristic of stem cells: commitment, resulting in loss of potential to follow more than one differentiation programs. Two models of stem cell differentiation are recognized. According to the first model, the differentiated progeny of a stem cell are committed, whereas in the second, the differentiated progeny remain able to revert to a dedifferentiated transit stem cell, postulated for crypt cells of the intestine (Potten C S and Loeffler M. Development 1990; 110(4): 1001-1020). Judging from the reversibility of BMEL cell differentiation, we suggest that BMEL cells represent the first model for a transit stem cell in the liver.

Three BMEL cell lines (9A1, 10B1 and 14B3) were further analysed because they contained palmate-like cells and displayed an uncoupled phenotype, expressing LETFs but only few liver functions (Spagnoli F M, et al Journal of Cell Biology 1998; 143(4): 1101-1112, Chaya D, et al Molecular and Cellular Biology 1997; 17(11): 6311-6320). These BMEL cells are bipotential: they differentiate as hepatocytes in aggregate culture or as bile duct cells in Matrigel. Their mixed morphology suggested that two cell types could be present. However, cloning revealed that the progeny were also of mixed morphology. When daughter clones were cultured in differentiating conditions, the bipotential state was shown to be heritable.

TABLE VI
BMEL cell daughter clones are bipotential

| | 9A1-1 | | 10B1-1 | | 14B3-1 | |
|---|---|---|---|---|---|---|
| | Basal | Induced | Basal | Induced | Basal | Induced |
| | | Matrigel | | Matrigel | | Matrigel |
| Thy-1 | − | + | − | + | − | + |
| GGT IV | − | + | − | + | − | + |
| CD34 | + | + | − | + | − | + |
| c-Kit | + | + | − | + | − | + |
| IB4 | + | + | + | + | + | + |
| Cx 43 | + | + | + | + | + | + |
| HNF6 | + | + | + | + | − | + |
| | | Aggre- | | Aggre- | | Aggre- |
| | | gates | | gates | | gates |
| AFP | + | ++ | + | ++ | + | ++ |
| Albumin | + | ++ | − | + | − | + |
| Apo AIV | + | ++ | − | + | − | + |
| ADH | − | + | − | + | − | + |
| Apo B | − | + | − | + | − | + |
| Aldo B | + | + | + | ++ | − | + |

−: no expression +: expressed ++: strongly expressed

### Example 2

One of the experimental mouse models that is used to study liver cell transplantation is the Albumin-urokinase Plasminogen Activator (Alb-uPA) transgenic mouse (Sandgren et al., 1991, Cell 66: 245-256). In these mice, the toxic enzyme uPA is expressed specifically in hepatocytes, which induces a progressive loss of these cells, and results in the death of the animal between 3-6 weeks post natum. However, as a rare event, a hepatocyte is able to excise the transgene. These hepatocytes then proliferate and form nodules which eventually repopulate the entire liver within 1 to 2 months, thus saving the animal (Sandgren et al., 1991, Cell 66: 245-256). The Alb-uPA mice have been successfully used to study the capacity of primary cultures from adult hepatocytes or embryonic hepatocytes to repopulate the liver (Rhim et al., 1994, Science 263: 1149-1152) (Weglarz et al., 2000, American Journal of Pathology 157: 1963-1974) (Cantz et al., 2003, American Journal of Pathology 162: 37-45). With these experiments, the authors have shown the ability of primary hepatocytes to participate in liver regeneration. However, no cell line, with the ability to participate in liver regeneration, had so far been described. A few studies have shown the homing of cells from cell lines to the liver (Suzuki et al., 2002, Journal of Cell Biology 156: 173-184) (Tanimizu et al., 2003, Journal of Cell Science 116: 17751786). However, there had been no proof of their participation in liver regeneration as witnessed by the formation of proliferating clusters of hepatocytes and the neo-formation of bile duct structures. We now show that BMEL cell lines injected into Alb-uPA mice are able to participate in liver regeneration by forming large clusters of hepatocytes and bile duct cells, and this for up to 8 weeks after cell injections. These results demonstrate that BMEL cells are stem cells, able to differentiate not only in vitro but also in vivo as hepatocytes and bile duct cells.

Materials and Methods

BMEL Cell Culture

BMEL and BMEL-GFP cell lines are cultured in basal culture medium which is composed of RPMI 1640 (Invitrogen), 10% fetal calf serum (Sigma), 50 ng/ml epidermal growth factor (PeproTech), 30 ng/ml insulin-like growth factor II (PeproTech), 10 μg/ml insulin (Roche) and antibiotics on Collagen type I (Sigma) coated dishes. Cells are dissociated with trypsin-EDTA and passaged every 3-4 days at a cell density of 8.6×10³ cells/cm². Cells are cultured in a humidified atmosphere with 7% CO₂ at 37° C.

BMEL Cell Line Transduction with the TRIP Vector Lentivirus: BMEL-GFP Cell Lines

Cell lines 9A1 and 14B3 were incubated overnight with 500 ng of p24 TRIP-GFP vector and 5 μg/ml DEAE Dextran in RPMI 1640 medium according to the established protocol (Zennou et al., 2000, Cell 101: 173-185). The next day the medium was changed to basal culture medium. In the following days the cells were expanded, FRCS analysis was performed to determine the percentage of cells that express GFP, and the cells were frozen at a density of 3-5×10⁶ cells per vial in 0.5 ml 10% DMSO 90% serum.

BMEL-GFP Cell Injection into Alb-uPA Scid Mice

BMEL-GFP cell lines were thawed and expanded for 2 days before injection. The cells were dissociated in trypsin-EDTA and single cell suspensions were counted and resuspended in Williams medium (Invitrogen) at a concentration of 1×10⁶ cells/ml. Alb-uPA Scid transgenic mice 3-5 weeks post nature were anesthetized. An incision was made to allow access to the spleen, into which were injected slowly 0.5×10⁶ cells. The mouse was suturized and maintained at 37° C. until the next day. The next day, and every week thereafter, the mice were subjected to an anti-macrophage treatment. All mice were maintained in a SPF environment with humane care.

Immunohistochemical Analysis of Liver Sections

Mouse livers were rapidly frozen in OCT compound (Sakura) and 10 μm serial cryostat sections through the entire liver were performed. The sections were fixed in 4% paraformaldehyde (Merck) for 15 minutes (min) at 20° C. (this temperature was used throughout the protocol). Between each step, sections were rinsed in PBS 1×. Sections were permeabilized in 0.1% triton (Sigma) for 10 min. The endogenous peroxidases were inhibited by incubation of the sections in 0.3% H₂O₂ for 5 min. A blocking step of 30 min in 10% goat serum was performed. The primary antibody, anti-GFP (Molecular Probes), anti-Albumin ( . . . ), anti-CK19 (Troma 3 a kind gift from R. Keinler, Max-Planck Institut of Immunobiology, Germany), or anti-DPPIV (CD26 Pharmingen) with 5% goat serum in PBS was incubated on the section for 2 hours. Sections were washed in PBS for 15 min before incubation with the appropriate secondary antibody: either goat anti-Rabbit conjugated to peroxidase (DAKO), or goat anti-Mouse conjugated to peroxidase (Caltag), or goat anti-Rat conjugated to peroxidase (Caltag) for 1 hour. Sections were washed for 15 min in PBS before revealing the presence of peroxidase with liquid DAB+ (3,3′diaminobenzidine) chromogen (DAKO) for 5 min. Lastly, the sections were counterstained using Mayer's Hematoxylin (Merck) and mounted in aqueous mounting medium (Shandon).

Results

Two BMEL cell lines (9A1 and 14B3) were transduced with the TRIP lentiviral vector in which the expression of the green fluorescent protein (GFP) is under control of the cytomegalovirus (CMV) promoter (Zennou et al., 2000). By FACS analysis we determined that the fraction of GFP expressing cells was around 70-90%. With further culture, this fraction diminished to approximately 50%. It is possible that the presence of GFP protein at high levels could be toxic for cells. This could be the case in our experiments since the GFP gene is driven by the strong promoter CMV. Thus the cells that do not express GFP could have a proliferative advantage over those that do express the protein.

Cells of the two lines were thawed and cultured 2 days before injection. Alb-uPA Scid mice 3-5 weeks after birth were anesthetized and 0.5×10⁶ cells were injected into the spleen. The Alb-uPA mice originate from crosses between two heterozygous mice, therefore the transgenic animals were recognizable by their “white liver” phenotype, as described by Sandgren et al. For each cell line (9A1-GFP and 14B3-GFP) numerous mice were injected. The surviving mice (19 out of 33 injected=57.6%) were sacrificed 3, 5, and 8 weeks after the operation (Table VII). The livers of the mice were dissected and serially sectioned for analysis.

TABLE VII

| | Mouse | | # of | Time of | | |
|---|---|---|---|---|---|---|
| Mouse | Pheno- | Cell line | cells | sacrifice after | | |
| # | type | injected | injected | injection | | Result |
| 14 | uPA | 9A1-GFP | 0.5 × 10⁶ | 3 | weeks | Hepatocytes |
| | Scid | | | | | and Bile duct |
| | | | | | | cells |
| 27 | uPA | 14B3-GFP | 0.5 × 10⁶ | 3 | weeks | Hepatocytes |
| | Scid | | | | | and Bile duct |
| | | | | | | cells |
| 23 | uPA | 9A1-GFP | 0.5 × 10⁶ | 5 | weeks | Hepatocytes |
| | Scid | | | | | and Bile duct |
| | | | | | | cells |
| 24 | uPA | 14B3-GFP | 0.5 × 10⁶ | 5 | weeks | Hepatocytes |
| | Scid | | | | | and Bile duct |
| | | | | | | cells |
| 25 | uPA | 9A1-GFP | 0.5 × 10⁶ | 8 | weeks | Hepatocytes |
| | Scid | | | | | and Bile duct |
| | | | | | | cells |
| 26 | uPA | 14B3-GFP | 0.5 × 10⁶ | 8 | weeks | No cells |
| | Scid | | | | | found |
| 28 | uPA | 9A1-GFP | 0.5 × 10⁶ | 4½ | weeks | Not |
| | Scid | | | | | determined |

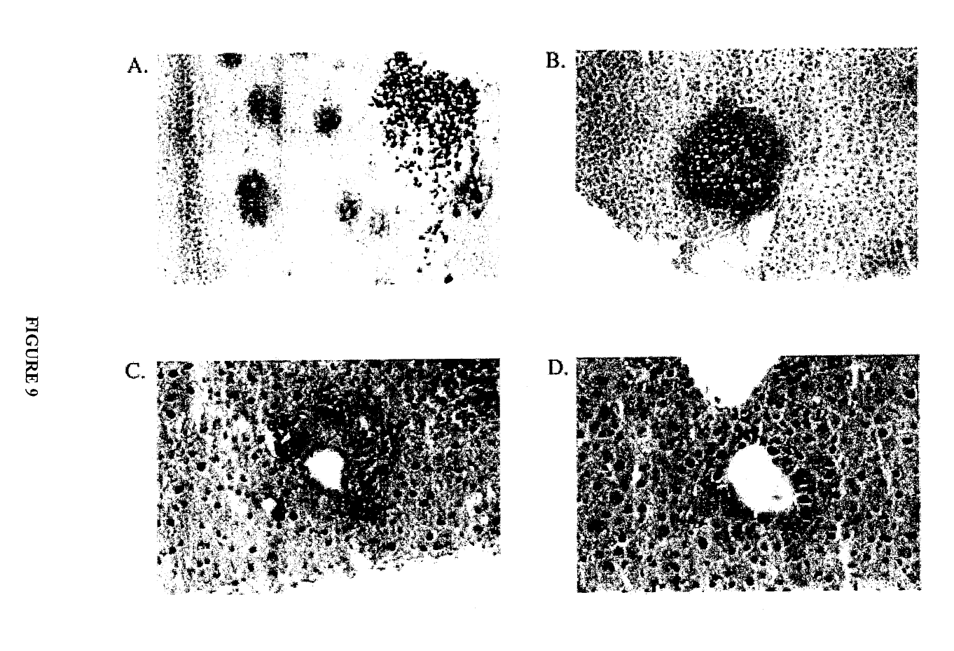

Although the GFP protein should be visible under the correct wavelength, the autofluorescence of liver tissue is too strong and precludes a definitive distinction of the injected cells. We therefore used immunohistochemistry to visualize the GFP expressing cells. Analysis of an Alb-uPA mouse 3 weeks after infusion with the cell line 9A1-GFP, subjected to immunohistochemistry, showed the presence of numerous clusters of GFP expressing cells (FIG. 9). These clusters of cells are localized randomly within the parenchyma, with no preference for perivenous or periportal zones. The clusters seem integrated within the hepatic plates and display the same morphology as the neighboring hepatocytes (FIG. 9 A, B). The presence of large groups of cells with a clonal aspect strongly implies that the cells have proliferated in vivo. Careful analysis of the liver sections also revealed that 9A1-GFP cells had participated in the formation of bile ducts (FIG. 9 C, D).

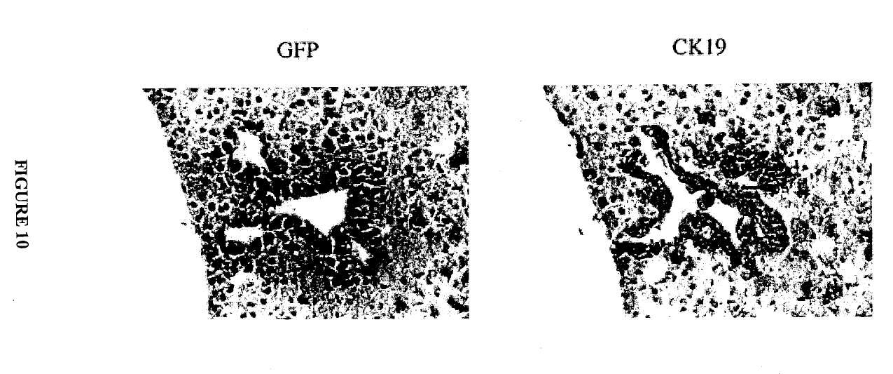

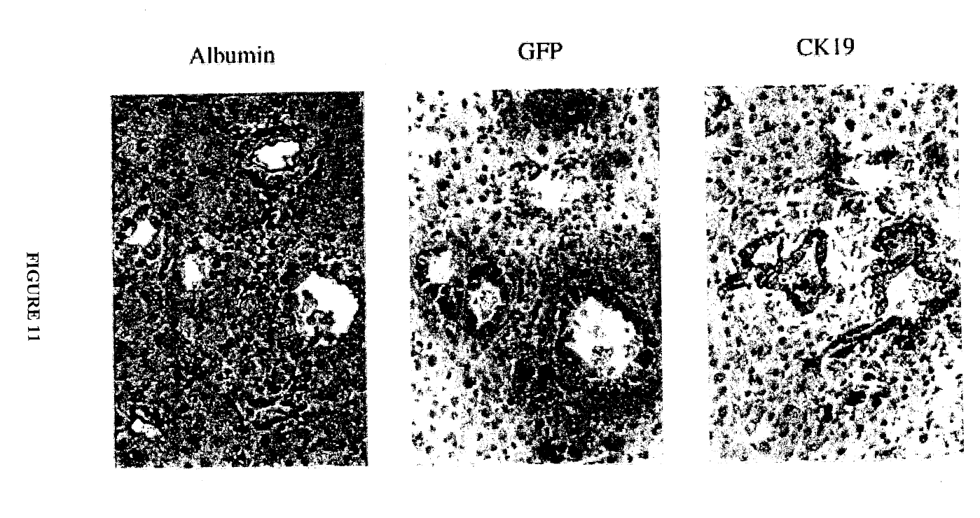

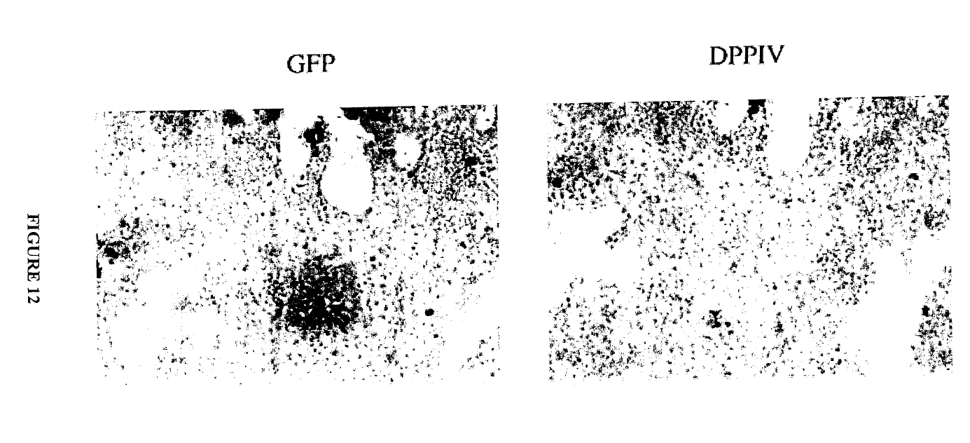

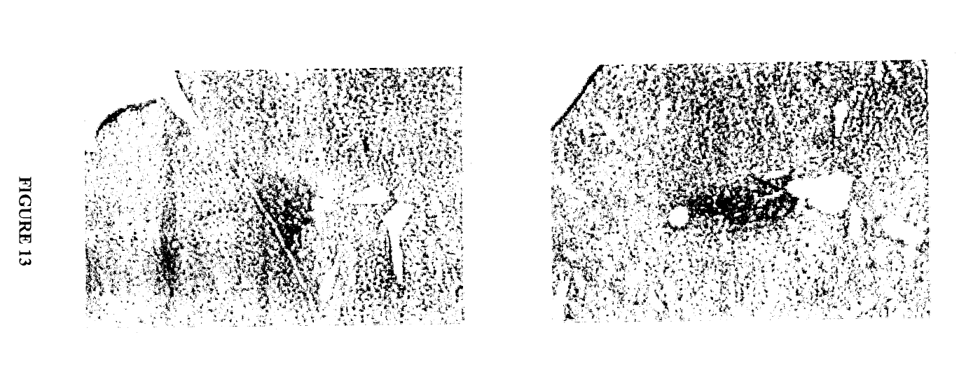

To determine whether the infused cells had differentiated in vivo into functional bile duct cells, immunohistochemistry was performed on adjacent serial sections using an antibody that recognizes the GFP protein and an antibody that recognizes the bile duct specific cytokeratin (CK) 19. The results showed that bile ducts consisting of 9A1-GFP cells also expressed CK19 (FIG. 10). A similar experiment using an antibody that recognizes Albumin, which is expressed by hepatocytes, revealed that 9A1-GFP cells differentiated as hepatocytes in vivo (FIG. 11). As expected, the hepatocyte clusters formed by 9A1-GFP cells do not contain CK19 (FIG. 11). The enzyme dipeptidyl peptidase IV (DPPIV) is localized specifically at the bile canaliculi of hepatocytes. An antibody that recognizes DPPIV was used to show that the 9A1-GFP cells expressed this marker in the liver parenchyma (FIG. 12). 9A1-GFP cells were still present 5 weeks after injection in the Alb-uPA mouse (FIG. 13).

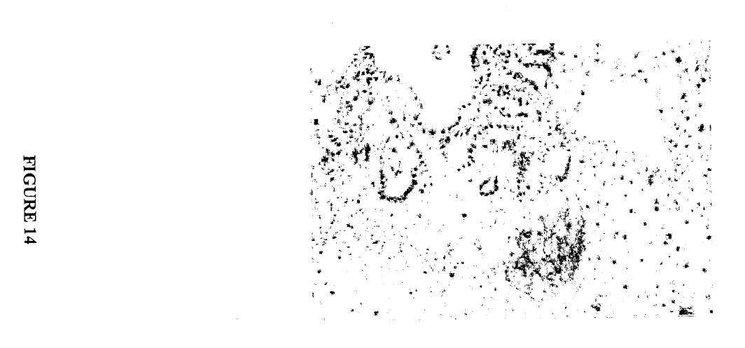

As has been previously indicated, the cell lines 9A1-GFP and 14B3-GFP consist of only 50% GFP expressing cells. Thus, using an antibody that recognizes GFP we were only visualizing half the fields of interest. To reveal all the infused cells, a different marker had to be used. The cell lines 9A1 and 14B3 are of genetic background C57Bl/6J/CBA, thus of MHC class I haplotype H₂B and H2K. The recipient Alb-uPA mice are of genetic background C57Bl/6JBalb/c, thus of MHC class I haplotype H₂B and H₂D. To recognize the infused cells, immunohistochemistry with an antibody that recognizes MHC class I haplotype H₂K was performed. The preliminary results show 14B3-GFP cells in the liver, which have differentiated as hepatocytes and bile duct cells, 5 weeks after injection (FIG. 14). Future experiments will compare adjacent serial sections analyzed by immunohistochemistry with an antibody that recognizes GFP, or haplotype H₂K, or haplotype H₂D.

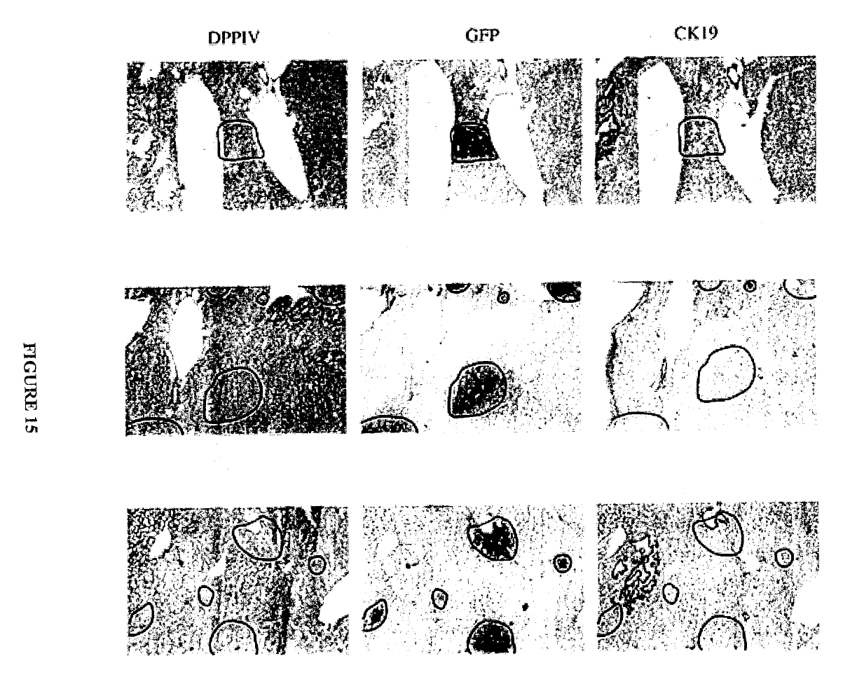

We have shown that cells of line 9A1-GFP differentiate as hepatocytes, bile duct cells and contribute to the liver regeneration of Alb-uPA mice. Similar results were obtained with cells of line 14B3-GFP: 5 weeks after infusion of the cells, the liver showed numerous clusters of GFP expressing cells (FIG. 15). Immunohistochemistry on adjacent serial sections revealed that the 14B3-GFP cells had integrated into the liver parenchyma, differentiated as hepatocytes and bile duct cells, as seen by the expression of marker genes DPPIV and CK19.

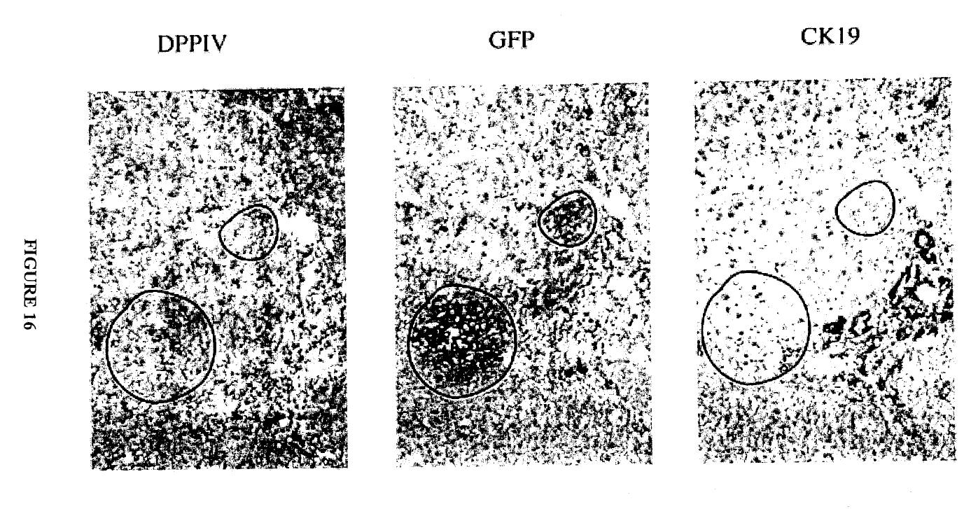

Finally, 8 weeks after cell injection, 9A1-GFP cells remain in the liver parenchyma, thus showing long-term engraftment (FIG. 16). These clusters of cells express the hepatocyte marker DPPIV.

Taken together, the results show that two embryonic cell lines, isolated from wild-type mice, differentiate in vitro and in vivo as hepatocytes and bile duct cells. The cells home to the liver, engraft, differentiate, and participate in liver regeneration in a model of continuous liver injury. The process is stable, since the cells are still present 8 weeks after they have been injected.

Obviously, numerous modifications and variations of the present invention are possible in light of the above teachings. It is therefore to be understood that within the scope of the appended claims, the invention may be practiced otherwise than as specifically described herein.

1. A cultured, immortalized, non-transformed mammalian hepatic cell line obtained by culturing cells obtained from a mammalian liver for at least one month under conditions suitable to obtain said cultured, immortalized, non-transformed mammalian hepatic cell.

2. The cultured cell line of claim 1, which is a stem cell.

3. The cultured cell line of claim 2, wherein the stem cell is bipotential.

4. The cultured cell line of claim 1, which is non-differentiated.

5. The cultured cell line of claim 1, which is differentiated.

6. The cultured cell line of claim 5, wherein the cells are differentiated into hepatocytes.

7. The cultured cell line of claim 5, wherein the cells are differentiated into bile ducts.

8. The cultured cell line of claim 1, wherein the mammal is a mouse.

9. The cultured cell line of claim 1, wherein the culturing time is at least 2 months.

10. The cultured cell of claim 1, wherein the culturing time is at least 3 months.

11. The cultured cell of claim 1, which is obtained from a mammalian embryonic liver.

12. A method of producing an immortalized, non-transformed mammalian hepatic cell line, comprising
obtaining a sample of liver tissue from a mammal, and
culturing the sample for at least one month under conditions suitable for the production of cultured, immortalized, non-transformed mammalian hepatic cell line.

13. The method of claim 12, wherein the immortalized, non-transformed mammalian hepatic cell line are stem cells.

14. The method of claim 13, wherein the stem cell is bipotential.

15. The method of claim 12, wherein the cells of the immortalized, non-transformed mammalian hepatic cell line are non-differentiated.

16. The method of claim 12, wherein the cells of the immortalized, non-transformed mammalian hepatic cell is differentiated.

17. The method of claim 12, wherein the mammal is a mouse.

18. The method of claim 12, wherein the culturing time is at least 2 months.

19. The method of claim 12, wherein the culturing time is at least 3 months.

20. The method of claim 12, wherein the liver tissue is embryonic liver tissue.

21. A method of generating liver tissue in a mammal, comprising producing the immortalized, non-transformed mammalian hepatic cell line of claim 1 and stimulating the hepatic cells of the cell line for a time and under conditions suitable to induce the hepatic cell to differentiate into liver tissue.

22. The cultured cell of claim 1, which is BMEL-14B3 deposited at the C.N.C.M on Oct. 3, 2003 under the accession number I-3100.

23. The cultured cell of claim 1, which is BMEL-9A1 deposited at the C.N.C.M on Oct. 3, 2003 under the accession number I-3099.

24. A method of generating liver tissue in a mammal, comprising injecting a composition comprising immortalized, non-transformed mammalian hepatic cells of the cell line according to claim 1 to generate liver tissue in said mammal.

25. A method of generating liver tissue in a mammal, comprising injecting a composition comprising immortalized, non-transformed mammalian hepatic cells of the cell line according to claim 2 to generate liver tissue in said mammal.

26. A method of generating liver tissue in a mammal, comprising injecting a composition comprising immortalized, non-transformed mammalian hepatic cells of the cell line according to claim 3 to generate liver tissue in said mammal.

27. A method of generating liver tissue in a mammal, comprising injecting a composition comprising immortalized, non-transformed mammalian hepatic cells of the cell line according to claim 4 to generate liver tissue in said mammal.

28. A method of generating liver tissue in a mammal, comprising injecting a composition comprising immortalized, non-transformed mammalian hepatic cells of the cell line according to claim 5 to generate liver tissue in said mammal.

29. A method of generating liver tissue in a mammal, comprising injecting a composition comprising immortalized, non-transformed mammalian hepatic cells of the cell line according to claim 6 to generate liver tissue in said mammal.

30. A method of generating liver tissue in a mammal, comprising injecting a composition comprising immortalized, non-transformed mammalian hepatic cells of the cell line according to claim 7 to generate liver tissue in said mammal.

31. A method of generating liver tissue in a mammal, comprising injecting a composition comprising immortalized, non-transformed mammalian hepatic cells of the cell line according to claim 8 to generate liver tissue in said mammal.

32. A method of generating liver tissue in a mammal, comprising injecting a composition comprising immortalized, non-transformed mammalian hepatic cells of the cell line according to claim 22 to generate liver tissue in said mammal.

33. A method of generating liver tissue in a mammal, comprising injecting a composition comprising immortalized, non-transformed mammalian hepatic cells of the cell line according to claim 23 to generate liver tissue in said mammal.

34. A method of identifying a compound which alters the development of the cultured cells of the cell line of claim 1, comprising
contacting the cultured cells with the compound; and
detecting at least one of an altered differentiation or development of the cultured cells into hepatocytes, bile duct, or both compared to the cultured cells not contacted with the compound.

35. A non-human mammal comprising the hepatic cells of the cell line according to claim 1.

36. A cultured, immortalized, non-transformed hepatic cell obtained from the cell line of claim 1.

37. A cultured, immortalized non-transformed mammalian hepatic cell obtained from the cell line produced by the method of claim 12.

38. A method of generating differentiated hepatocytes, bile ducts, or both, comprising producing the immortalized, non-transformed mammalian hepatic cell of claim 1 and stimulating the hepatic cell for a time and under conditions suitable to induce the hepatic cell to differentiate into hepatocytes, bile ducts or both.

39. The cultured cell line of claim 1, wherein the hepatic cells are transduced.

40. The method of claim 12, wherein the hepatic cells of the cell line are transduced.

41. The method of claim 21, wherein the hepatic cells of the cell line are transduced.

42. The method of claim 24, wherein the hepatic cells of the cell line are transduced.

43. The method of claim 34, wherein the hepatic cells of the cell line are transduced.

44. The non-human mammal of claim 34, wherein the hepatic cells of the cell line are transduced.

45. The hepatic cell of claim 36, which is transduced.

46. The method of claim 38, wherein the hepatic cells of the cell line are transduced.
